(19) United States
(12) Patent Application Publication
(10) Pub. No.: US 2023/0330232 A1
(43) Pub. Date: Oct. 19, 2023

(54) SELF-ASSEMBLED COMPLEX CONTAINING IRON ION

(71) Applicant: Korea University Research and Business Foundation, Seoul (KR)

(72) Inventors: Heemin KANG, Seoul (KR); Yu-Ri KIM, Seoul (KR); Gun-Hyu BAE, Seoul (KR); Sung-Gue LEE, Siheung-si (KR); Na-Yeon KANG, Seoul (KR); Sun-Hong MIN, Suwon-si (KR); Seong-Yeol KIM, Seoul (KR)

(73) Assignee: Korea University Research and Business Foundation, Seoul (KR)

(21) Appl. No.: 17/989,940

(22) Filed: Nov. 18, 2022

(30) Foreign Application Priority Data
Apr. 13, 2022 (KR) 10-2022-0046023
Aug. 5, 2022 (KR) 10-2022-0097806

(51) Int. Cl. A61K 47/02; A61K 9/00; A61K 45/06

(52) U.S. Cl. CPC A61K 47/02; A61K 9/0009; A61K 45/06

(57) ABSTRACT

Disclosed is a self-assembled complex containing an iron ion that includes an iron ion; and at least one ligand, where the iron ion and the ligand are reversibly self-assembled or self-disassembled. The iron ion and the ligand are self-assembled to form an assembly structure at least part of which is round shaped.

### CROSS-REFERENCE TO RELATED APPLICATIONS

This application claims priority to and the benefit of Korean Patent Application No. 10-2022-0046023 filed on Apr. 13, 2022 and Korean Patent Application No. 10-2022-0097806 filed on Aug. 5, 2022, in the Korean Intellectual Property Office, the entire contents of which are incorporated herein by reference.

### BACKGROUND OF THE INVENTION

### (A) Field of the Invention

The present invention relates to a self-assembled complex containing an iron ion, and particularly to a self-assembled complex containing an iron ion in which the iron ion and a ligand can be reversibly self-assembled and self-disassembled.

### (B) DESCRIPTION OF THE RELATED ART

A drug delivery system (DDS) is defined as a formulation for effectively delivering a therapeutic substance at a minimum amount required for healing a disease by minimizing the side effects of the existing drugs and optimizing the efficacy and effects of the therapeutic substance.

The field of drug delivery system enables the development of new formulations of the existing drugs with low costs in a short period of time and is recognized as the core of the next-generation bio industry. Therefore, research studies on the development of new drug delivery systems using biopolymers and synthetic polymers with various functions and performances are being actively conducted.

The conventional drug delivery systems make the use of artificially synthetized materials such as self-assembled complexes using diphosphate and a metal ion, which are synthetic materials not found in the human body, or Fe₃O₄ that causes a risk of toxicity in the body.

Such conventional drug delivery systems have an issue of toxicity due to the use of substances not found in the human body, involve the delivery of growth factors, proteins, etc., eventually not achieving good price competitiveness, and do not allow a sustained release under control, so they are limited to drug delivery over a short period of time only.

Besides, the drug delivery system is supposed to deliver a drug to a desired target site of action over a prolonged or given period of time so as to reduce the side effects of the drug and raise the efficacy to the maximum.

Therefore, there is a need to develop a drug delivery system that is harmless with little toxicity relative to the existing technologies by using natural substances found in human body, allows a sustained release of drugs, molecules or active ingredients under control, and enables an efficient delivery of drugs to a desired target site of action.

### SUMMARY OF THE INVENTION

### Technical Problem

It is an object of the present invention to provide a self-assembling complex containing an iron ion. The self-assembling complex is capable of delivering active ingredients to a desired target site of action in a living body through reversible self-assembly and self-disassembly.

### Technical Solution

In one aspect of the present invention, the embodiments of the present invention may include a self-assembled complex containing an iron ion that includes an iron ion; and at least one ligand, where the iron ion and the ligand are reversibly self-assembled or self-disassembled. The iron ion and the ligand may be self-assembled to form an assembled structure at least part of which is round shaped.

In an embodiment, the self-assembled complex may further include an active ingredient, where the active ingredient may be supported in the self-assembled complex.

In an embodiment, the active ingredient may be at least any one selected from the group consisting of adenosine, guanosine, uridine, cytidine, doxorubicin, a drug, a protein, an organic acid, an organic base, a fragrance, and a dye.

In an embodiment, the active ingredient may be released through a self-disassembly of the self-assembled complex, the active ingredient being released continuously over a release time.

In an embodiment, the release time may be 1 to 90 days.

In an embodiment, the ligand may include at least any one of phosphate and phosphonate.

In an embodiment, the ligand may be at least any one selected from the group consisting of AMP, ADP, ATP, TMP, TDP, TTP, CMP, CDP, CTP, GMP, GDP, GTP, UMP, UDP, UTP, DNA, RNA, AEP (2-aminoethylphosphonic acid), TNA (threose nucleic acid), GNA (glycol nucleic acid), HNA (1,5-anhydrohexitol nucleic acid), ANA (1,5-anhydroatritol nucleic acid), FANA (2′-deoxy-2′-fluoroarabino nucleic acid), and CeNa (cyclohexenyl nucleic acid).

In an embodiment, the ligand may include a combination of AMP and ATP, where the AMP and the ATP are included at a molar concentration ratio of 2:1 to 1:2.

In an embodiment, when the ligand includes at least any one of ADP and ATP, the self-assembled complex may be hydrophilic; and when the ligand includes AMP, the self-assembled complex may be hydrophobic.

In an embodiment, the ligand may be at least any one selected from a group consisting of AMP, ADP, a mixed ligand of ADP and ATP, and a mixed ligand of AMP and ATP. The self-assembled complex may be spherically shaped, and a plurality of the self-assembled complex may stick together to form a two-dimensional plate structure or a three-dimensional aggregate.

In an embodiment, the two-dimensional plate structure may spin in an applied magnetic field.

In an embodiment, the three-dimensional aggregate may include pores formed therein.

In an embodiment, at least any one of the two-dimensional plate structure and the three-dimensional aggregate may have cells supported therein to enable a deliver of the cells into a living body.

In an embodiment, the self-assembled complex may be formed by at least any one of π-π interaction and hydrogen bonding between the adjacent ligands; or coordination bonding between the iron ion and the ligand.

In an embodiment, the iron ion and the ligand may be included at a molar concentration ratio of 5:1 to 1:1.

In an embodiment, the rate of self-disassembly may vary by the type of the ligand.

In an embodiment, the self-assembled complex may be self-disassembled over 1 to 90 days.

In an embodiment, the self-disassembly may be accelerated under conditions with at least any one of a chelating agent, a strong acid, and a strong base. The condition with a strong acid may have a pH of 2 to 5, and the condition with a strong base may have a pH of 9 to 12.

In an embodiment, the self-disassembly may be accelerated to have a duration of 0.5 second to one minute.

In an embodiment, the self-assembled complex may be paramagnetic, where the magnetic moment may increase with an increase in the size of the self-assembled complex.

In an embodiment, when a magnetic field is applied to the self-assembled complex in vivo or in vitro, the larger the self-assembled complex, the more quickly it spreads.

In an embodiment, the self-assembled complex may be used as at least any one selected from a substance delivery carrier, a cell culture medium, a T2 contrast agent, and an artificial bone.

### Effects of Invention

According to the present invention as described above, it is possible to provide a self-assembled complex containing an iron ion. The self-assembled complex is capable of delivering an active ingredient into a living body through reversible self-assembly and self-disassembly.

### BRIEF DESCRIPTION OF DRAWINGS

FIG. 1 is a schematic diagram showing the formation of a Fe-ATP self-assembled complex according to an embodiment of the present invention.

FIGS. 2, 3 and 4 present analytical results for the characteristics of a Fe-ATP self-assembled complex, a Fe-ADP self-assembled complex and a Fe-AMP self-assembled complex, respectively.

FIG. 5 presents images showing that an iron ion and ATP are self-assembled.

FIG. 6 presents the results of an observation that the self-assembled complex is self-disassembled under conditions with EDTA.

FIG. 7 presents analytical results for the characteristics of a self-assembled complex of which the size is adjusted by the concentrations of the iron ion and ATP.

FIG. 8 is a graph showing the reversible magnetic properties (paramagnetism) of a Fe-ADP self-assembled complex.

FIG. 9 shows that a self-assembled complex moves in the direction of applying a permanent magnet.

FIG. 10 shows that a self-assembled complex having a 2D plate structure spins in an applied magnetic field.

FIG. 11 shows that a self-assembled complex having a 3D scaffold structure is moving in an applied magnetic field.

FIG. 12 shows that the speed of motion is varied as a function of the size for the self-assembled complex placed in an applied magnetic field.

FIG. 13 presents experimental results for the performance of a Fe-ATP self-assembled complex as an MRI contrast agent.

FIG. 14 presents analytical results for the magnetic properties of a self-assembled complex introduced into a living body and then placed in a magnetic field applied from the outside of the living body.

FIGS. 15 and 16 present experimental results for the self-disassembly of a self-assembled complex under conditions with EDTA in vivo or in vitro.

FIG. 17 presents experimental results for the self-disassembly of a self-assembled complex having a 2D plate structure under conditions with EDTA.

FIG. 18 is a schematic diagram showing the self-assembly of an iron ion, ATP and doxorubicin (DOX).

FIG. 19 present observation results for the release time of doxorubicin supported in the self-assembled complex.

FIG. 20 is a schematic diagram showing a self-assembly of an iron ion, ATP and a drug molecule (triamcinolone acetonide).

FIG. 21 presents the observation results for the release time of drug molecules (triamcinolone acetonide) supported in the self-assembled complex.

### DETAILED DESCRIPTION OF THE EMBODIMENTS

The details of other embodiments are included in the detailed description and drawings.

The advantages and features of the present invention and methods to achieve these will be more clearly understood from the following detailed description taken in conjunction with the accompanying drawings. However, the present invention is not limited to the embodiments disclosed below, but may be implemented in a variety of different forms. Unless otherwise specified in the following description, all numbers, values and/or expressions indicating the components, reaction conditions, and contents of components in the present invention are modified in all instances by the term “about”, as these numbers are inherently approximations reflecting the various uncertainties of measurements arising out of obtaining such values, among others. Also, in disclosing numerical ranges in this description, such ranges are continuous and inclusive of all values from the minimum to the maximum, unless otherwise specified. Moreover, when such ranges refer to integers, all integers ranging from the minimum to the maximum are included, unless otherwise specified.

When a range is defined for a variable in the present invention, the variable will be understood to include all values within the defined range, including the recited endpoints of the range. For example, a range of “5 to 10” includes the values of 5, 6, 7, 8, 9, and 10, as well as any sub-ranges, such as 6 to 10, 7 to 10, 6 to 9, and 7 to 9. It will be also construed as including any value between integers that are appropriate for the scope of the defined range, such as 5.5, 6.5, 7.5, 5.5 to 8.5, and 6.5 to 9. For example, a range of “10 to 30%” includes values of 10%, 11%, 12%, 13%, etc. and all integers up to 30%, as well as any sub-range, such as 10 to 15%, 12 to 18%, and 20 to 30%, and any value between reasonable integers within the scope of the defined range, such as 10.5%, 15.5%, and 25.5%.

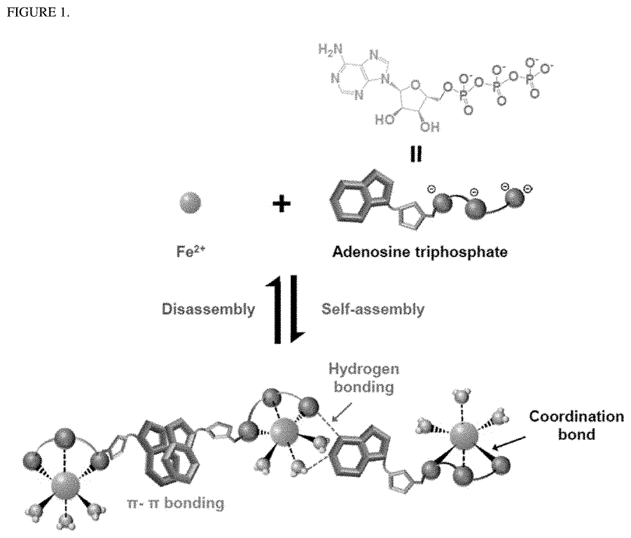

FIG. 1 is a schematic diagram showing an iron-containing self-assembled complex according to an embodiment of the present invention.

FIG. 1 shows self-assembly and self-disassembly of an iron ion (Fe²⁺) and adenosine triphosphate (ATP) in a schematic manner. The iron ion can form a coordination bond with the phosphate moiety of ATP and water to produce a metal complex. The aromatic ring of the adenine moiety forms a π-π interaction. In addition, the nitrogen of the adenine moiety and the water molecule bound to the iron ion can form a hydrogen bond.

In that manner, the self-assembled complex may be formed to include coordination bonding, π-π interaction, and hydrogen bonding, at least one of which can be used to form the self-assembled complex.

In one aspect of the present invention, the embodiments of the present invention may include a self-assembled complex containing an iron ion that includes an iron ion; and at least one ligand, where the iron ion and the ligand are reversibly self-assembled or self-disassembled. The ligand and the iron ion may be self-assembled to form an assembled structure at least part of which is round shaped.

The self-assembled complex may further include an active ingredient, and the active ingredient may be supported in the self-assembled complex. The active ingredient may become supported inside the self-assembled complex during a self-assembly of the self-assembled complex.

The self-assembled complex may release the active ingredient when it is self-disassembled. It is therefore possible to control the release time and rate of the active ingredient by adjusting the self-disassembly time of the self-assembled complex. The self-assembled complex may have the self-disassembly rate variable by the type of the ligand. The self-assembled complex may be self-disassembled at a defined rate sequentially from the surface thereof, which allows the active ingredient to be continuously released for the self-disassembly time of the self-assembled complex. In other words, the active ingredient may be released in a sustained manner over a release time.

The self-disassembly time of the self-assembled complex, i.e., the release time of the active ingredient may range from 1 day to 90 days. In the environment that promotes the self-disassembly of the self-assembled complex, the release time of the active ingredient may range from 0.5 second to one minute; or from 0.5 second to 10 seconds.

The active ingredient may be a substance capable of being supported in the self-assembled complex and released during the self-disassembly of the self-assembled complex. For example, the active ingredient may be at least any one selected from the group consisting of adenosine, guanosine, uridine, cytidine, doxorubicin, a drug, a protein, an organic acid, an organic base, a fragrance, and a dye. The drug may be triamcinolone acetonide.

The ligand may be a substance capable of forming a coordination bond with the iron ion and participating in the self-assembly. A bonding may occur between the two adjacent ligands to form a metal complex.

The ligand may include at least any one of phosphate and phosphonate. The phosphate or phosphonate may be a moiety capable of forming a coordination bond with the iron ion.

For example, the ligand may be at least any one selected from the group consisting of adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), DNA, RNA, 2-aminoethylphosphonic acid (AEP), threose nucleic acid (TNA), glycol nucleic acid (GNA), 1,5-anhydrohexitol nucleic acid (HNA), 1,5-anhydroatritol nucleic acid (ANA), 2′-deoxy-2′-fluoroarabino nucleic acid (FANA), and cyclohexenyl nucleic acid (CeNA).

The iron ion may form a coordination bond with an electron-rich moiety, such as the phosphate moiety of the ligand, to make a metal complex.

At least part of the self-assembled complex may be round shaped. Further, the self-assembled complex may be spherically shaped. The self-assembled complex may be formed with bondings between a plurality of the iron ions and the ligands. The iron ion and the ligand may form a bonding at a molar concentration ration of 5:1 to 1:1, preferably 1:1.

The self-assembled complex may be spherically shaped or at least partly round shaped as formed by at least any one of coordination bonding, π-π interaction, and hydrogen bonding between the iron ion and the ligand that are adjacent to each other. In the self-assembled complex, the iron ion and the ligand can be densely adhered to each other by the interactions or bonding. Hence, the self-assembled complex may be formed by dense aggregation of the iron ion and the ligand.

Depending on the type and mixing ratio of the ligand, the self-assembled complex may be formed more densely or loosely. The mixing ratio may be a molar concentration ratio.

One or two types of the ligand may be used to form a self-assembled complex.

When the ligand includes, for example, diphosphate or triphosphate, the self-assembled complex may be hydrophilic. With the ligand including triphosphate, the self-assembled complex may be negatively charged with a valence of 2. With the ligand including diphosphate, the self-assembled complex may be negatively charged with a valence of 1. When the ligand includes diphosphate or triphosphate, it may mean that the ligand is at least any one of ADP, ATP, TDP, TTP, CDP, CTP, GDP, GTP, UDP, and UTP.

When the ligand includes, for example, monophosphate, the self-assembled complex may be hydrophobic. With the ligand including monophosphate, the self-assembled complex may be neutral. When the ligand includes monophosphate, it may mean that the ligand is at least any one of AMP, TMP, CMP, GMP, and UMP.

Besides, the ligand may include a combination of AMP and ATP, in which case the self-assembled complex may be hydrophilic. With the ligand including a combination of AMP and ATP, the AMP and the ATP may be included at a molar concentration ratio of 2:1 to 1:2, preferably 1:1.

When the ligand is ATP, the self-assembled complex may be formed in the shape of a sphere with a uniform diameter. In this case, the self-assembled complexes may not aggregate. The diameter of the self-assembled complex may vary by the concentrations of the ligand and the iron ion, but it may be uniform at same concentrations of the ligand and the iron ion.

When the ligand is AMP, a mixed ligand of AMP and ADP, or a mixed ligand of AMP and ATP, the self-assembled complex may be formed in the shape of a sphere. Further, the self-assembled complexes may form an aggregate. The aggregate of the self-assembled complexes may not have a specific shape. In this case, a plurality of the self-assembled complexes may form an aggregate having a three-dimensional shape.

The self-assembled complex may be self-disassembled in a spontaneous manner. As for the self-disassembly, the self-assembled complex may be self-disassembled over 1 to 90 days in vivo or in vitro. As the self-assembled complex is self-disassembled, it may release the iron ions or the ligands, and the active ingredient as well in the case that the active ingredient is supported in the self-assembled complex.

The self-assembled complex may release the active ingredient in the manner of a sustained release.

The self-disassembly rate of the self-assembled complex may vary by the type of the ligand. The electric charge and properties of the self-assembled complex may vary by the type and mixing ratio of the ligand. This characteristic may determine the degree of self-assembly of the self-assembled complex. It is therefore possible to control the disassembly rate by adjusting the type and mixing ratio of the ligand.

When the ligand is AMP, for example, one phosphate group forms a coordination bond with an iron ion (Fe²⁺) to make the self-assembled complex neutral. In this regard, the iron ion may be additionally bound to a water molecule. The neutral self-assembled complex may be packed more densely due to its hydrophobic properties, in which case self-disassembly may proceed more slowly.

When the ligand is ADP, for example, two phosphate groups form coordination bonds with an iron ion to make the self-assembled complex negatively charged with a valence of 1. But, this case may cause the complex packed more loosely (with lower density) due to the lower valence than the case of the self-assembled complex negatively charged with a valence of 2. Besides, this case may lead to forming the complex packed more loosely than the case of using AMP as the ligand. Therefore, the self-assembled complex having ADP as the ligand may be self-disassembled more quickly.

When the ligand is AMP, ADP, ATP, and a mixed ligand of AMP+ATP, for example, the rate of self-disassembly may increase in the order of AMP, ATP, AMP+ATP, and ADP.

The rate of self-disassembly may correspond to the release rate of at least any one of the active ingredient, the iron ion, and the ligand.

The self-assembled complex may be self-disassembled over 1 to 90 days. But, it may take less than 3 days for the self-assembled complex to be self-disassembled in the case of self-disassembly taking place in vivo.

Besides, the self-disassembly may proceed faster when the self-assembled complex is placed under specific conditions. The self-assembled complex may undergo a self-disassembly accelerated under conditions with at least any one of a chelating agent, a strong acid, and a strong base.

The chelating agent can form a bond with an iron ion in place of the ligand to promote the self-disassembly of the self-assembled complex. The chelating agent may be a substance with higher affinity to the iron ion than the ligand, and include, for example, EDTA.

The condition with a strong acid or a strong base can also accelerate the cleavage of the bond between the iron ion and the ligand and hence the self-disassembly of the self-assembled complex as well. For example, the strong acid may be hydrochloric acid (HCl), and the strong base may be sodium chloride (NaCl).

For example, the condition with a strong acid may have a pH of 2 to 5, and the condition with a strong base may have a pH of 9 to 12. Preferably, the condition with a strong acid may have a pH of 3 to 4, and the condition with a strong base may have a pH of 10 to 11.

The accelerated self-disassembly of the self-assembled complex may take 0.5 to 10 seconds.

The self-assembled complex may have paramagnetic properties due to the iron ion contained therein. The magnetic moment may increase with an increase in the size of the self-assembled complex. Hence, the larger the self-assembled complex, the greater the attractive force (or repulsive force) induced by the magnetic field applied to the self-assembled complex. In other words, when a magnetic field is applied to the self-assembled complex in vivo or in vitro, the larger self-assembled complex may be forced to spread faster.

In addition, as the self-assembled complex has magnetic properties only with an external magnetic field applied thereto, it is easy to control the self-assembled complex in vivo and in vitro.

Due to the paramagnetic properties, the self-assembled complex may be available as an MRI contrast agent, preferably as a T2 contrast agent.

When the ligand is AMP, the self-assembled complex may be formed more densely and assembled more firmly to have a three-dimensional form. With the ligand being AMP, the self-assembled complex may be assembled to form a two-dimensional plate structure or a three-dimensional scaffold structure and thus available as a material for artificial bones.

Like the self-assembled complex, the two- or three-dimensional structure made of the self-assembled complex may exhibit self-assembling/disassembling and paramagnetic properties. When placed in a magnetic field, the two-dimensional structure may spin due to its paramagnetic properties.

The two-dimensional plate structure or the three-dimensional scaffold structure may contain pores formed therein. The pores may support at least any one of cells, drugs, and other substances to deliver. It is therefore possible to deliver substances, such as cells, into a living body by recognition of the structure in vivo.

For example, a mold is made using PMMA particles and removed as melting. Then, the voids deprived of the PMMA can be filled with a substance to deliver, so the mold is available as a carrier for delivery of the substance into a living body.

The self-assembled complex may be used as at least any one of a substance delivery carrier, a cell culture medium, a T2 contrast agent, and an artificial bone.

Hereinafter, a description will be given as to the examples and comparative examples of the present invention. The following examples are given only to illustrate preferred embodiments of the present invention and are not to be construed to limit the scope of the present invention.

### EXAMPLES

### 1. Example 1 (Fe-AMP)

A Fe-AMP self-assembled complex was fabricated as follows.

A 20 mM FeCl₂ solution and a 20 mM AMP solution were separately prepared using deionized (DI) water. 1 mL of the FeCl₂ solution was added to 1 mL of the AMP solution (at the same concentration), and the two solutions were blended on a vortex mixer. The mixed solution thus obtained was incubated at 37° C. for 12 hours.

Once a spontaneous self-assembly took place, the solution containing Fe-AMP self-assembled particles was centrifuged at 10,000 rpm for 5 minutes.

In order to remove the unreacted reagent, the supernatant of the solution was discarded, and the remaining solution was mixed with additional DI water and centrifuged. This procedure was repeated twice.

After washing, the Fe-AMP self-assembled particles thus collected were dispersed in 2 mL of DI water.

### 2. Example 2 (Fe-ADP)

A Fe-ADP self-assembled complex was fabricated as follows.

A 20 mM FeCl₂ solution and a 20 mM ADP solution were separately prepared using deionized (DI) water. 1 mL of the FeCl₂ solution was added to 1 mL of the ADP solution (at the same concentration), and the two solutions were blended on a vortex mixer. The mixed solution thus obtained was incubated at 37° C. for 12 hours. Once a spontaneous self-assembly took place, the solution containing Fe-ADP self-assembled particles was centrifuged at 10,000 rpm for 5 minutes.

In order to remove the unreacted reagent, the supernatant of the solution was discarded, and the remaining solution was mixed with additional DI water and centrifuged. This procedure was repeated twice.

After washing, the Fe-ADP self-assembled particles thus collected were dispersed in 2 mL of DI water.

### 3. Example 3 (Fe-ATP)

A size-controllable Fe-ATP self-assembled complex was fabricated as follows.

Using deionized (DI) water, a FeCl₂ solution and an ATP solution were prepared with different concentrations. Fe-ATP self-assembled particles having a size of 100 nm, 300 nm, 1,000 nm, 1,500 nm, and 3,000 nm can be produced from the FeCl₂ and ATP solutions each having a concentration of 2.5 mM, 5 mM, 10 mM, 20 mM, and 30 mM, respectively.

1 mL of the FeCl₂ solution was added to 1 mL of the ATP solution (at the same concentration), and the two solutions were blended on a vortex mixer. The mixed solution thus obtained was incubated at 37° C. for 12 hours. Once a spontaneous self-assembly took place, the solution containing Fe-ATP self-assembled particles was centrifuged at 10,000 rpm for 5 minutes.

In order to remove the unreacted reagent, the supernatant of the solution was discarded, and the remaining solution was mixed with additional DI water and centrifuged. This procedure was repeated twice.

After washing, the Fe-ATP self-assembled particles thus collected were dispersed in 2 mL of DI water. 100 μL of 100 mM EDTA solution was gently added to the dispersed solution. In a few seconds, the self-disassembly of the Fe-ATP particles was observed.

TABLE 1

| | Iron ion | ATP | | |
|---|---|---|---|---|
| | concentration | concentration | Particle size | |
| Example 3-1 | 2.5 mM | 2.5 mM | 100 | nm |
| Example 3-2 | 5 mM | 5 mM | 300 | nm |
| Example 3-3 | 10 mM | 10 mM | 1,000 | nm |
| Example 3-4 | 20 mM | 20 mM | 1,500 | nm |
| Example 3-5 | 30 mM | 30 mM | 3,000 | nm |

### 4. Example 4 (Fe-(AMP+ATP))

A Fe-(AMP+ATP) self-assembled complex was fabricated as follows.

A FeCl₂ solution, an AMP solution, and an ATP solution each with a concentration of 10 mM were separately prepared using deionized (DI) water. 1 mL of the FeCl₂ solution, 1 mL of the AMP solution and 1 mL of the ATP solution were blended on a vortex mixer. The mixed solution thus obtained was incubated at 37° C. for 12 hours.

Once a spontaneous self-assembly took place, the solution containing Fe-(AMP+ATP) self-assembled particles was centrifuged at 10,000 rpm for 5 minutes.

In order to remove the unreacted reagent, the supernatant of the solution was discarded, and the remaining solution was mixed with additional DI water and centrifuged. This procedure was repeated twice.

After washing, the Fe-(AMP+ATP) self-assembled particles thus collected were dispersed in 2 mL of DI water.

### Experiment Methods

1. Scanning Electron Microscopy (SEM) Imaging

SEM (FEI, Quanta 250 FEG) imaging was performed in order to determine the morphological properties of a self-assembled complex. The self-assembled complex prepared was collected, dried in a vacuum oven at room temperature, and platinum-plated for 90 seconds, followed by the SEM imaging.

2. Vibrating Sample Magnetometry (VSM) Measurement

In order to analyze the magnetic properties of a self-assembled complex, VSM (Tecnai 20, FEI, USA) measurement was performed by applying a magnetic field between −19000 Oe and 19000 Oe at room temperature. After normalization to the dry weight of the samples, the magnetic moments induced across the samples were provided by the reversible hysteresis loops.

3. Transmission Electron Microscopy (TEM) Imaging and Energy-Dispersive X-Ray Spectroscopy (EDS) Mapping

TEM imaging and EDS mapping (Tecnai 20, FEI, USA) were performed to determine the size and elemental composition of a self-assembled complex.

4. In-Situ Optical Microscopy Imaging

In-situ optical microscopy imaging (Nikon Eclipse Ts2) was performed to observe the spontaneous self-assembly or self-disassembly of the self-assembled complex and determine the moving speed and direction of the self-assembled complex in an applied magnetic field.

5. Synthesis of 2D Fe-AMP Plate

20 mL of a solution containing 500 mM AMP in DI water, 20 mL of a solution containing 500 mM FeCl₂ in DI water, and 20 mL of a solution containing rhodamine (Rhodamine 6G, CAS: 989-38-8, Sigma-Aldrich) in DI water were prepared for synthesis of 2D Fe-AMP plates.

First, 2 mL of the rhodamine solution was added to 20 mL of the 500 mM FeCl₂ solution. After adding 20 mL of the 500 mM AMP solution, the mixed solutions were blended on a vortex mixer at room temperature for one hour. In collect a Fe-AMP self-assembled complex with rhodamine supported therein, the mixed solution was centrifuged at 12,000 rpm for 10 minutes, and the supernatant was discarded. In order to remove the unreacted reagent, 400 mL of DI water was added to 40 mL of the rhodamine-supporting Fe-AMP self-assembled complex, followed by centrifugation at 12,000 rpm for 10 minutes. This procedure was repeated four times.

Subsequently, the rhodamine-supporting Fe-AMP self-assembled complex was dried in an oven at 37° C. for 5 days. The dry self-assembled complex was collected and ground using mortar. Using a hand press, about 10 mg of the self-assembled complex was pressed into a 2D Fe-AMP plate (13 mm in diameter, 1 mm in height).

The 2D plate was placed in a PBS solution, followed by applying a magnetic field for observation of the plate. An optical fluorescence microscope (Nikon Eclipse Ts2) was used to observe the movement of the 2D plate according to the position of the permanent magnet and the self-disassembly of the 2D plate induced by adding 1 mL of DI water.

6. Preparation of 2D Fe-AMP Cell Sheet

An experiment was performed for the culture of cells on 2D Fe-AMP.

Prior to seeding cells, the 2D Fe-AMP plate prepared in the above Experiment 5 was cut into pieces and put in a 48-well cell culture plate. The cell culture plate was placed in 50 μL of a high glucose DMEM (Dulbecco's modified eagle medium) culture medium containing 10% heat-inactivated fetal bovine serum, 4 mM L-glutamine and 50 U/mL penicillin/streptomycin and incubated at 37° C. under 5% CO₂ conditions for 24 hours. The culture medium was renewed every 8 hours.

Subsequently, human mesenchymal stem cells (hMSCs, Lonza, PT-2501, passage #5) were seeded on the 20 Fe-AMP plate at a density of about 2×10⁵ cells/cm² and cultured for 48 hours, followed by collecting the 2D Fe-AMP cell plate (2D Fe-AMP plate with the cells attached thereto). The 2D Fe-AMP cell plate thus collected was turned over and placed in a PBS solution. Immediately after being placed in 1 mL of a solution containing 1M EDTA in DI water, the 2D Fe-AMP cell plate was self-disassembled, which was observed through an optical fluorescence microscope (Nikon Eclipse Ts2).

7. Preparation of 3D Fe-AMP Bone Scaffold

A poly(methyl methacrylate) (PMMA) leaching method was used to synthesize a 3D Fe-AMP macroporous bone scaffold.

First, 300 mg of PMMA (125-150 μm in diameter; Bangs Laboratories, BB05N) was filled in a cylindrical polyethylene mold (8 mm in diameter). 200 μL of a solution containing 2M FeCl₂ in DI water was added to the mold, followed by gently blending on a vortex mixer for 5 minutes. The mold containing the resultant mixture was sealed and kept at 25° C. for one day.

After that, the 3D Fe-AMP aggregate was separated from the mold and immersed in 50 mL of dichloromethane (DCM) for 3 days under shaking condition (100 rpm) to leach the PMMA. The DCM was renewed every 24 hours. After 3 days, the 3D Fe-AMP aggregate was immersed in 50 mL of DI water for 30 minutes and then dried out.

The 3D Fe-AMP aggregate was cut to a height of 1 to 1.5 cm and sterilized by UV radiations for 30 minutes prior to its use for bone healing treatment.

8. Experiment for Doxorubicin Release

An experiment for doxorubicin release was performed to evaluate the substance delivery performance of the self-assembled complex. Solutions of FeCl₂, ATP, ADP, or AMP in DI water (each having a same concentration of 100 mM) were prepared for synthesis of Fe-ATP, Fe-ADP, Fe-AMP, or Fe-(ATP+AMP, 1:1) self-assembled complexes containing doxorubicin (Boryung Pharmaceutical, Adimycin, Item Standard Cod: 199001007), respectively.

2 mL of the 100 mM ATP (or ADP/AMP/ATP+AMP) solution and 2 mL of the 100 mM FeCl₂ solution were added to 6 mL of a doxorubicin solution (2 g/L in concentration), followed by blending on a vortex mixer. In order to remove the unreacted reagent, the supernatant was discarded, and DI water was added. The remaining solution was then centrifuged twice.

After washing, the Fe-ATP, Fe-ADP, Fe-AMP, or Fe-(ATP+AMP, 1:1) self-assembled particles were collected and dispersed in 1 mL of a PBS solution (pH 7.4). The dispersed solution was transferred to a dialysis bag (SnakeSkin™ Dialysis Tubing, 7 kDa MWCO). The dialysis bag containing the Fe-ATP, Fe-ADP, Fe-AMP, or Fe-(ATP+AMP, 1:1) self-assembled complex was placed in a vial, and 4 mL of a PBS solution (pH 7.4) was added to the vial to a total volume of 5 mL. On days 1, 3 and 7, the supernatant was removed from the vial with the dialysis bag containing the Fe-ATP, Fe-ADP, Fe-AMP, or Fe-(ATP+AMP, 1:1) self-assembled complex and measured in regards to the concentration of the released doxorubicin.

If necessary, additional PBS solution was added to the vial to maintain the total volume of 5 mL. The sample solutions were filtered through a 0.5 μm syringe filter (Advantec), and the supernatant was analyzed by UV-Vis spectroscopy.

9. Ingredients for Experiments

The substances used in the above experiments were as follows.
ATP: Adenosine 5′-triphosphate disodium salt hydrate (99%, Sigma-Aldrich, 5 g, cat. No. A26209)
AMP: Adenosine-5′-monophosphate disodium salt (J61643, Alfa Aesar)
ADP: Adenosine 5′-diphosphate sodium salt (A2754, Sigma-Aldrich)
FeCl₂: Iron(II) chloride (97%, Sigma-Aldrich, 25 g, cat. No. 372870)
EDTA: Ethylenediaminetetraacetic acid (EDTA, 99.5%, Junsei, 500 g, cat. No. 17385S0401)
Rhodamine: Rhodamine 6G (CAS: 989-38-8, Sigma-Aldrich)
PMMA: PMMA (125 to 150 μm in diameter; Bangs Laboratories, BB05N)
Doxorubicin: Boryung Pharmaceutical, Adimycin (Item Standard Cod: 1990 01007)

### Experimental Examples

1. Morphological Analysis

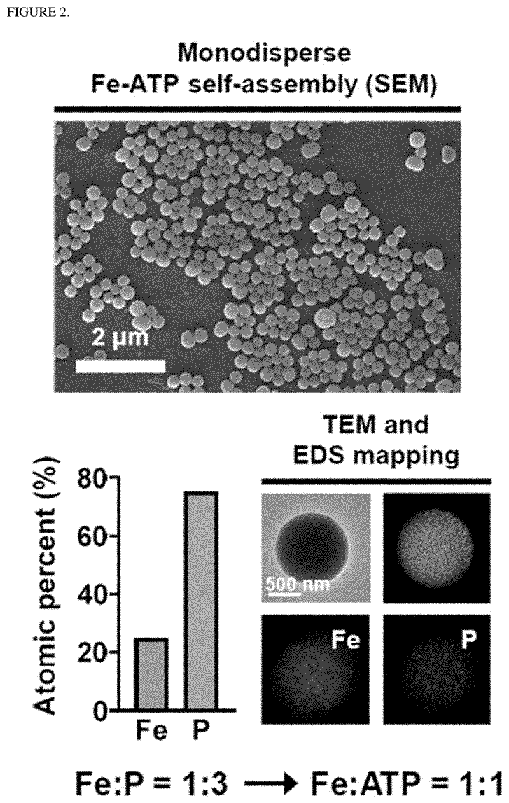

FIGS. 2, 3 and 4 are diagrams analyzing the shape and components of the Fe-AMP, Fe-ADP and Fe-ATP self-assembled complexes, respectively. EDS mapping was performed for iron (Fe) and phosphorous (P) elements.

The SEM image in the upper part of FIG. 2 shows that the Fe-ATP self-assembled complex has a uniform size. As shown in the TEM and EDS images, the complex contains iron (Fe) and phosphorous (P) elements (in the right lower part of FIG. 2), and iron ion (Fe²⁺) and ATP are also contained at a ratio of 1:1.

FIG. 3 is a diagram for the Fe-ADP self-assembled complex, showing that the complex contains iron (Fe) and phosphorous (P) and that the iron ion and ADP are contained at a ratio of 1:1. As can be seen from the TEM and EDS images, there is a little three-dimensional aggregate of the self-assembled complexes.

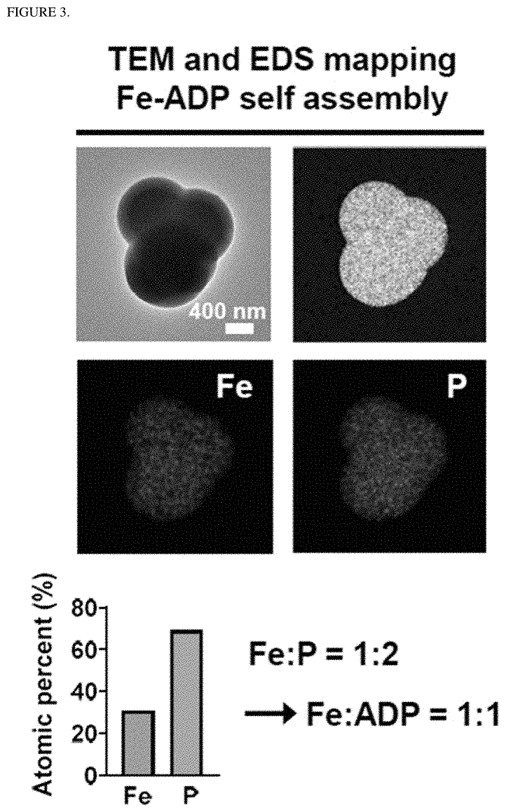

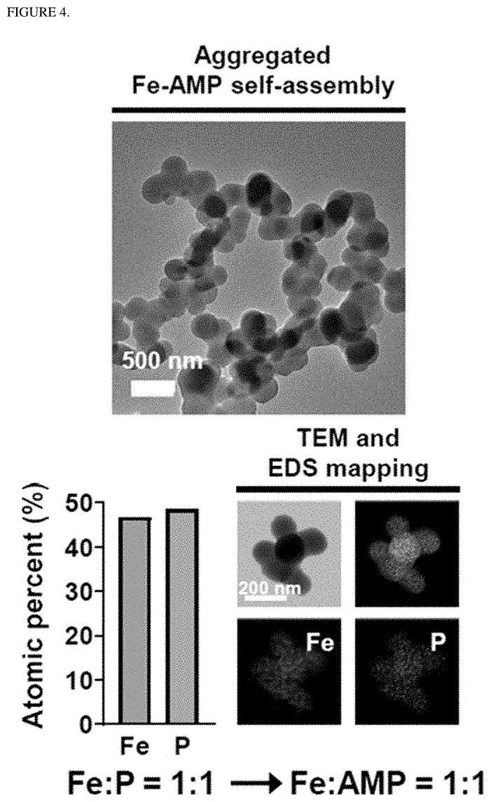

FIG. 4 is a diagram for the Fe-AMP self-assembled complex, showing that the complex contains iron (Fe) and phosphorous (P) and that the iron ion and AMP are contained at a ratio of 1:1. According to the TEM and EDS images, however, there are lots of three-dimensional aggregates of the self-assembled complexes.

2. Observation of Self-Assembly and Self-Disassembly

Self-assembly and self-disassembly of an iron ion and a ligand were observed, where the ligand was ATP.

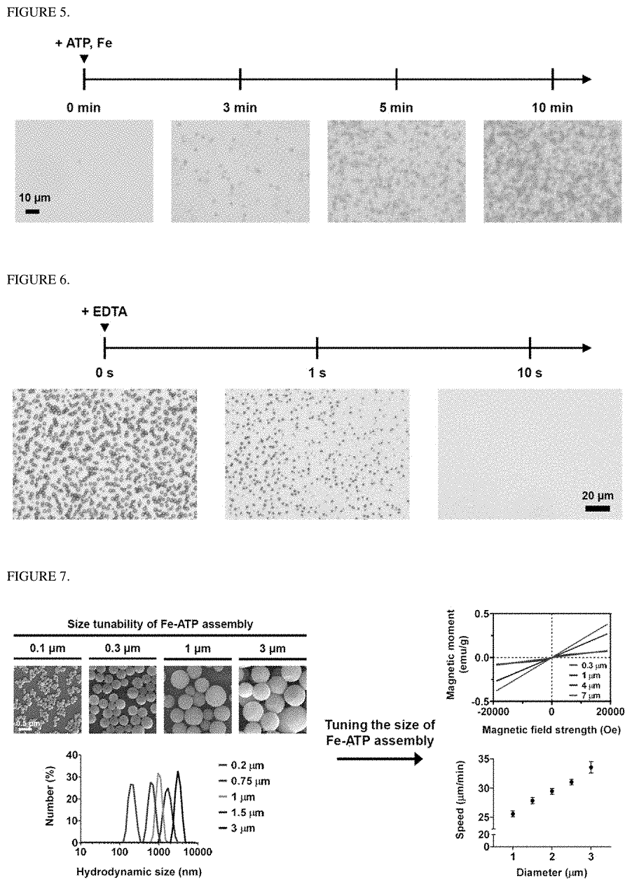

Referring to FIG. 5, self-assembled particles began to appear as a result of a spontaneous self-assembly of the iron ion and ATP in about 3 minutes and self-assembled complexes were formed rapidly in 10 minutes. Referring to FIG. 6, the self-assembled complexes were disassembled under conditions with EDTA and all disappeared in 10 seconds.

3. Analysis on Properties of Self-Assembled Complex as a Function of Size

Referring to FIG. 7, a self-assembly complex was prepared with its size varied by adjusting the concentrations of the iron ion and ATP and then analyzed in regards to the properties. Fe-ATP self-assembled complexes with different sizes displayed different magnetic properties (paramagnetism) and moved at different speeds in an applied magnetic field. FIG. 8 shows the reversible magnetic property (paramagnetism) of a Fe-ADP self-assembled complex.

According to the SEM images of FIG. 7, the size of the spherical self-assembled complex increased with an increase in the concentrations of the iron ion and ATP. But, the self-assembled complex had an almost constant size when formed at the constant concentrations of the iron ion and ATP. As can be seen from the VSM measurement results, the magnetic monument increased with an increase in the size of the self-assembled complex.

Besides, the larger the size of the self-assembled complex, the faster the self-assembled complex was attracted to the permanent magnet. This is because the magnetic moment increases with an increase in the size of the self-assembled complex.

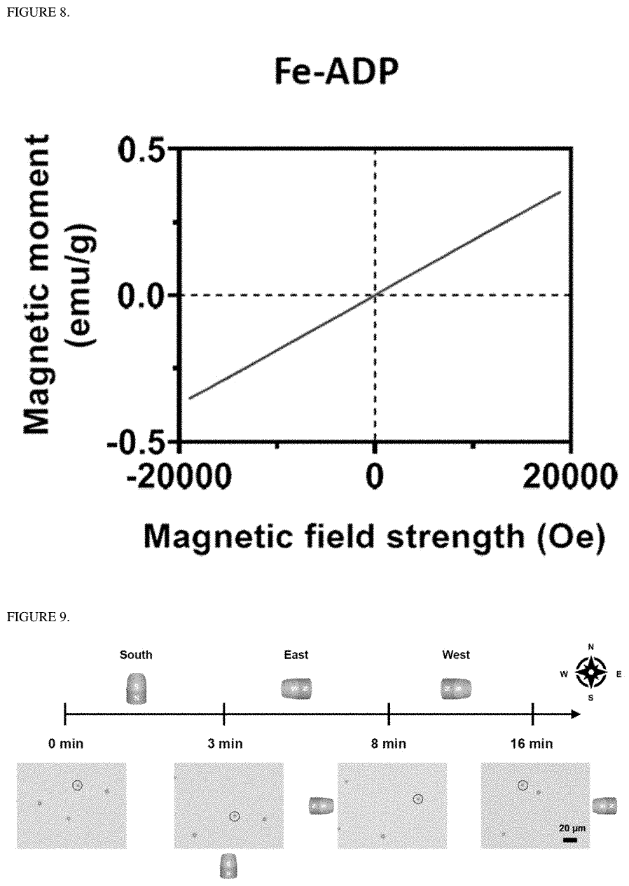

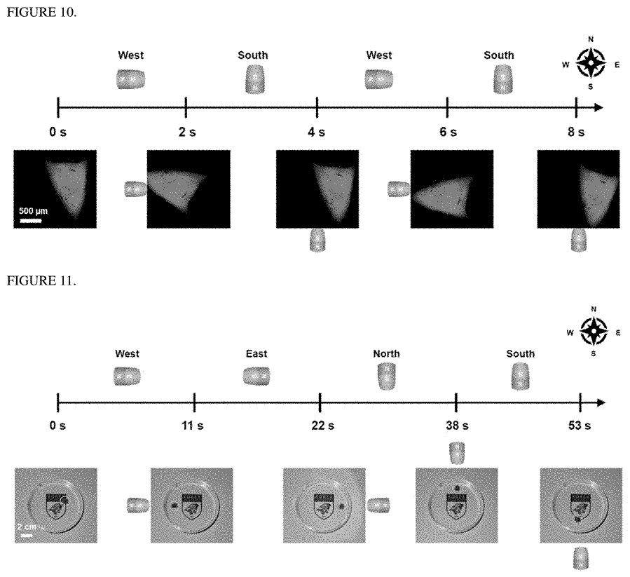

Referring to FIGS. 9, 10 and 11, the self-assembled complex or its structure moved in the direction of applying a permanent magnet, which implied that the self-assembled complex was paramagnetic. FIG. 10 presents the experimental results for the 2D plate structure of Fe-AMP, showing that the 2D plate spun in an applied magnetic field. FIG. 11 presents the experimental results for the 3D scaffold structure of Fe-AMP, showing that the 3D scaffold structure was paramagnetic regardless of the shape and size of the self-assembled complex.

FIG. 12 presents images showing a comparison of the speed at which a self-assembled complex was attracted to a permanent magnet, where the self-assembled complex had a size of 1.5 μm, 2 μm, 3 μm, or 3.5 μm. When it comes to a comparison of the moving speed between the self-assembled complexes with sizes of 1.5 μm and 3.5 μm, which have the largest difference in size, the self-assembled complex with a size of 3.5 μm had a moving speed at least twice higher than the self-assembled complex with a size of 1.5 μm. It can be seen from this result that the magnetic moment increases with an increase in the size of the self-assembled complex.

4. Performance as MRI Contrast Agent

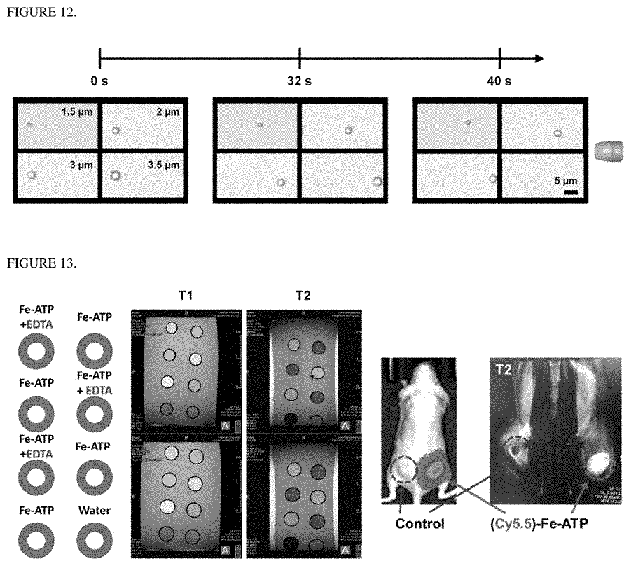

An experiment was performed to evaluate the performance of the Fe-ATP self-assembled complex as an MRI contrast agent. The experimental results are presented in FIG. 13. The experiment was conducted under the conditions of Fe-ATP and Fe-ATP+EDTA, where water was used as a control.

The Fe-ATP self-assembled complex was not observed in the TI images, but showed MRI signals in the T2 images. In the presence of EDTA, the self-assembled complex was disassembled, resulting in no T2 signal in the images. Without EDTA, the self-assembled complex displayed the performance of Fe and ATP as a T2 contrast agent.

Referring to FIG. 13, illustrating an injection of the self-assembled complex into a mouse, the Cy5.5-containing self-assembled complex (red circle) caused a T2 image to appear in contrast to the control (green circle). It was therefore confirmed that the self-assembled complex acted stably in vivo.

5. Evaluation on Properties of Self-Assembled Complex In Vivo

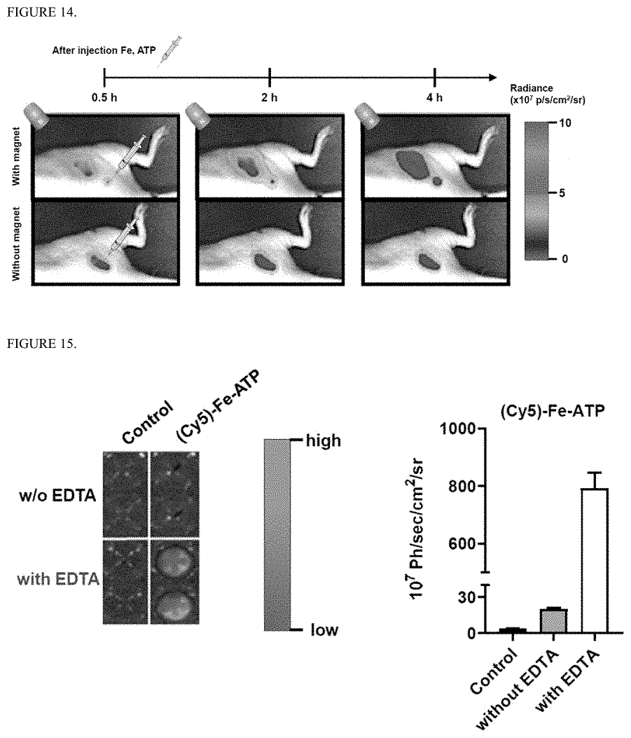

After injection of the self-assembled complex in vivo, a magnetic field was externally applied to evaluate the magnetic properties of the self-assembled complex. The results are presented in FIG. 14. Here, the ligand of the self-assembled complex was ATP, and a whole-body fluorescence microscope was used to take images. From the images, with the red part indicating the location of the self-assembled complex, it can be seen that the self-assembled complex spread as gradually moving towards the permanent magnet. Therefore, it is possible to control the site of action of the self-assembled complex in vivo by externally applying a magnetic field.

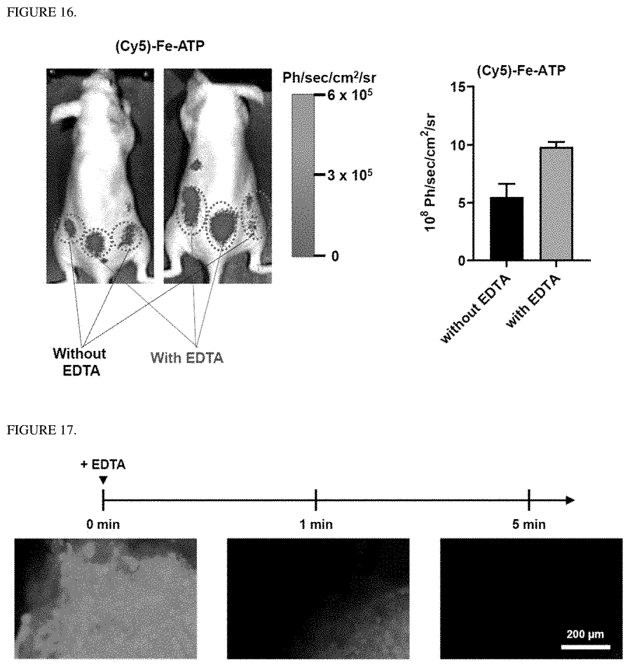

FIGS. 15 and 16 show an experiment on the disassembly of the self-assembled complex in vivo. The experiment was conducted using a Cy5-encapsulated Fe-ATP self-assembled complex under conditions with and without EDTA. The Fe-ATP complex spread as being self-disassembled only in the presence of EDTA injection (FIG. 15). According to the bioluminescence images of a mouse with the Fe-ATP complex injection, albeit showing fluorescence regardless of the presence of EDTA, the appearance of fluorescence was observed over a wider range under conditions with EDTA injection due to dispersion of the Fe-ATP self-assembled complex (FIG. 16).

FIG. 17 shows the two-dimensional plate structure of the rhodamine-containing Fe-AMP self-assembled complex undergoing a rapid self-disassembly under conditions with EDTA injection. The self-assembled complex was mostly disassembled about one minute after the EDTA injection and disappeared 5 minutes after the injection.

6. Drug Delivery Experiment

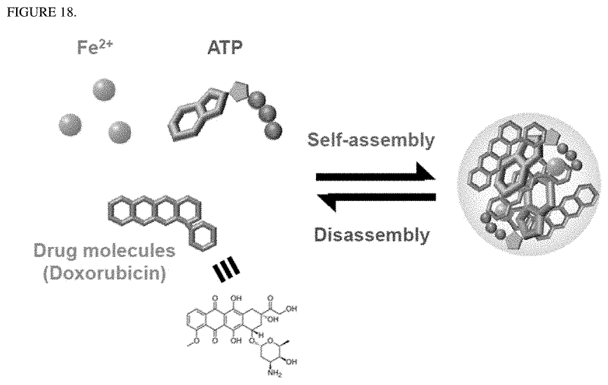

An experiment was performed in regards to the release of a drug supported in a self-assembled complex. The experimental procedure and results are shown in FIGS. 18 to 21.

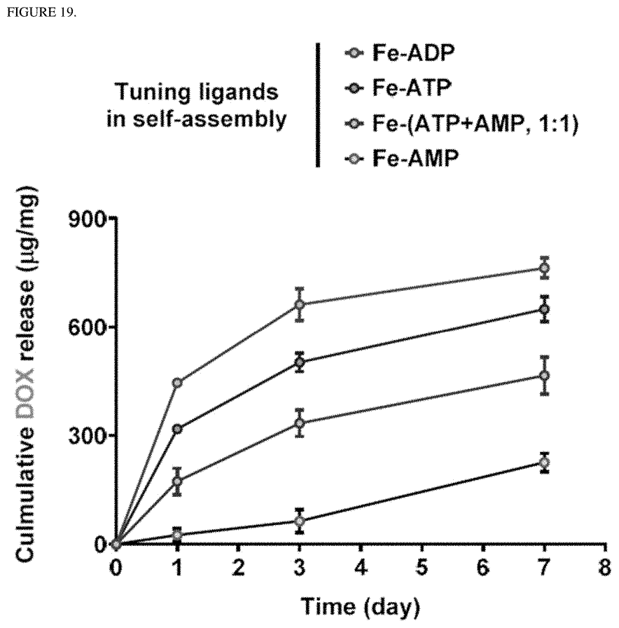

FIGS. 18 and 19 present a schematic diagram and results of an experiment using doxorubicin. The schematic diagram shows the doxorubicin being supported as penetrating into the dense structure of the self-assembled complex during a self-assembly of the iron ion and the ligand. FIG. 19 is a graph showing the release time of doxorubicin according to the type of the ligand. The conditions other than the type of the ligand were all the same. The cumulative release amount of doxorubicin from the Fe-AMP complex that was most densely packed was the least, whereas the doxorubicin was most rapidly released from the Fe-ADP complex that was most loosely packed. It is therefore possible to control the release time of a drug in vivo by adjusting the type of the ligand.

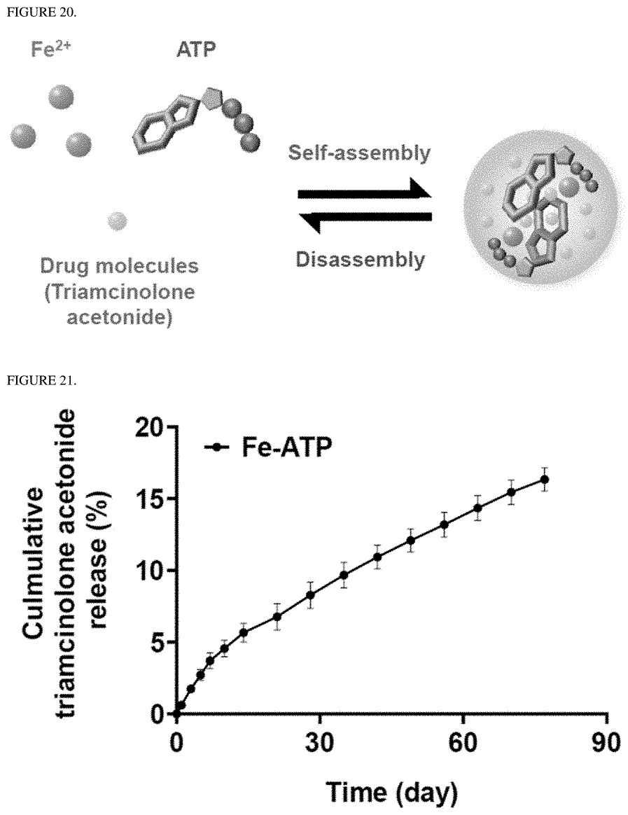

FIGS. 20 and 21 present a schematic diagram and results of an experiment using triamcinolone acetonide. With the progress of self-disassembly, the self-assembled complex released triamcinolone acetonide, resulting in the cumulative release amount of the drug increasing even after about 70 days, which can be seen from the graphs.

It should be apparent to those skilled in the present invention that many modifications and variations are possible without departing from the concept or essential features of the present invention. Therefore, the foregoing examples are to be construed as merely illustrative, and not limitative of the present invention. The scope of the present invention is defined by the appended claims rather than the detailed description of the present invention and should be construed as including all changes or modifications derived from the meaning and scope of the claims and their equivalents.

What is claimed is:

1. A self-assembled complex containing an iron ion, comprising:
an iron ion; and
at least one ligand,
wherein the iron ion and the ligand are reversibly self-assembled or self-disassembled,
wherein the iron ion and the ligand are self-assembled to form an assembled structure at least part of which is round shaped.

2. The self-assembled complex containing an iron ion according to claim 1, further comprising an active ingredient,
wherein the active ingredient is supported in the self-assembled complex.

3. The self-assembled complex containing an iron ion according to claim 2, wherein the active ingredient is at least any one selected from the group consisting of adenosine, guanosine, uridine, cytidine, doxorubicin, a drug, a protein, an organic acid, an organic base, a fragrance, and a dye.

4. The self-assembled complex containing an iron ion according to claim 2, wherein the active ingredient is released through self-disassembly of the self-assembled complex,
the active ingredient being released continuously over a release time.

5. The self-assembled complex containing an iron ion according to claim 4, wherein the release time is 1 to 90 days.

6. The self-assembled complex containing an iron ion according to claim 1, wherein the ligand is at least any one selected from the group consisting of AMP, ADP, ATP, TMP, TDP, TTP, CMP, CDP, CTP, GMP, GDP, GTP, UMP, UDP, UTP, DNA, RNA, AEP (2-aminoethylphosphonic acid), TNA (threose nucleic acid), GNA (glycol nucleic acid), HNA (1,5-anhydrohexitol nucleic acid), ANA (1,5-anhydroatritol nucleic acid), FANA (2′-deoxy-2′-fluoroarabino nucleic acid), and CeNa (cyclohexenyl nucleic acid).

7. The self-assembled complex containing an iron ion according to claim 1, wherein the ligand comprises a combination of AMP and ATP, wherein the AMP and the ATP are included at a molar concentration ratio of 2:1 to 1:2.

8. The self-assembled complex containing an iron ion according to claim 1, wherein when the ligand comprises at least any one of ADP and ATP, the self-assembled complex is hydrophilic,
wherein when the ligand comprises AMP, the self-assembled complex is hydrophobic.

9. The self-assembled complex containing an iron ion according to claim 1, wherein the ligand is at least any one selected from a group consisting of AMP, ADP, a mixed ligand of ADP and ATP, and a mixed ligand of AMP and ATP,
wherein the self-assembled complex is spherically shaped,
wherein a plurality of the self-assembled complex stick together to form a two-dimensional plate structure or a three-dimensional aggregate.

10. The self-assembled complex containing an iron ion according to claim 9, wherein the two-dimensional plate structure spins when placed in an applied magnetic field.

11. The self-assembled complex containing an iron ion according to claim 9, wherein the three-dimensional aggregate includes pores formed therein.

12. The self-assembled complex containing an iron ion according to claim 9, wherein at least any one of the two-dimensional plate structure and the three-dimensional aggregate has cells supported therein to enable a deliver of the cells into a living body.

13. The self-assembled complex containing an iron ion according to claim 1, wherein the self-assembled complex is formed by at least any one of π-π interaction and hydrogen bonding between the ligands; or coordination bonding between the iron ion and the ligand.

14. The self-assembled complex containing an iron ion according to claim 1, wherein the iron ion and the ligand are included at a molar concentration ratio of 5:1 to 1:1.

15. The self-assembled complex containing an iron ion according to claim 1, wherein the rate of self-disassembly varies by the type of the ligand.

16. The self-assembled complex containing an iron ion according to claim 1, wherein the self-assembled complex is self-disassembled over 1 to 90 days.

17. The self-assembled complex containing an iron ion according to claim 1, wherein self-disassembly is accelerated under conditions with at least any one of a chelating agent, a strong acid, and a strong base,
wherein the condition with a strong acid has a pH of 2 to 5,
wherein the condition with a strong base has a pH of 9 to 12.

18. The self-assembled complex containing an iron ion according to claim 14, wherein self-disassembly is accelerated to have a duration of 0.5 second to one minute.

19. The self-assembled complex containing an iron ion according to claim 1, wherein the self-assembled complex is paramagnetic,
wherein magnetic moment increases with an increase in the size of the self-assembled complex.

20. The self-assembled complex containing an iron ion according to claim 1, wherein when a magnetic field is applied to the self-assembled complex in vivo or in vitro, the larger the self-assembled complex, the more quickly it spreads.
